(19) United States
(12) Patent Application Publication
(10) Pub. No.: US 2026/0241171 A1
(43) Pub. Date: Aug. 20, 2026

(54) DEVICES, SYSTEMS, AND METHODS FOR TREATING SLEEP DISORDERED BREATHING

(71) Applicants: XII Medical, Inc., Union City, CA (US); The Cleveland Clinic Foundation, Cleveland, OH (US)

(72) Inventors: Anthony V. Caparso, North Ridgeville, OH (US); Francis A. Papay, Westlake, OH (US)

(21) Appl. No.: 19/159,534

(22) PCT Filed: Mar. 1, 2024
(86) PCT No.: PCT/US2024/018000

§ 371 Date: Aug. 25, 2025

Related U.S. Application Data
(60) Provisional application No. 63/449,599, filed on Mar. 2, 2023.

(51) Int. Cl. A61N 1/36; A61N 1/05

(52) U.S. Cl. CPC A61N 1/36078; A61N 1/0556; A61N 1/0558

(57) ABSTRACT

Devices, systems, and methods for treating sleep apnea are disclosed herein. According to some embodiments, the present technology includes an implantable device comprising an electronics package and first and second elongate leads electrically coupled to the electronics package. Each of the first and second leads can comprise (a) a first conductive region configured to be implanted at a first treatment site at an under-chin region of the patient's head, where the first conductive region is configured to deliver a first electrical signal to the first treatment site to thereby activate the genioglossus muscle, and (b) a second conductive region configured to be implanted at a second treatment site at a neck region of the patient, where the second conductive region is configured to deliver a second electrical signal to the second treatment site to thereby activate an infrahyoid strap muscle.

### CROSS-REFERENCE TO RELATED APPLICATION(S)

The present application claims the benefit of priority to U.S. Provisional Patent Application No. 63/449,599, filed Mar. 2, 2023, titled DEVICES, SYSTEMS, AND METHODS FOR TREATING SLEEP DISORDERED BREATHING, which is incorporated by reference herein in its entirety.

### TECHNICAL FIELD

The present technology relates to devices, systems, and methods for treating sleep disordered breathing.

### BACKGROUND

Sleep disordered breathing (SDB) occurs when there is a partial or complete cessation of breathing that occurs many times throughout the night. Obstructive sleep apnea (OSA) is a type of SDB that involves cessation or significant decrease in airflow in the presence of breathing effort. It is the most common type of SDB and is characterized by recurrent episodes of upper airway collapse during sleep inducing repetitive pauses in breathing followed by reductions in blood oxygen saturation or neurologic arousal. The pathophysiology of OSA can involve factors such as craniofacial anatomy, airway collapsibility, and neuromuscular control of the upper airway dilator musculature. Electromyogram studies have shown that the tonic and phasic activity of the pharyngeal airway dilatory muscles (such as the genioglossus muscle) is progressively reduced from wakefulness to non-rapid eye movement to rapid eye movement.

Continuous positive airway pressure (CPAP) therapy is the frontline treatment for OSA. CPAP therapy utilizes machines, generally including a flow generator, tubing, and a mask designed to deliver a constant flow of air pressure to keep the airways continuously open in patients with OSA. However, the success of CPAP therapy is limited by compliance with reported rates ranging from 50% to 70%. Hypoglossal nerve stimulation (HNS) has now been established as an effective form of therapy for patients with obstructive sleep apnea (OSA) who are unable to tolerate positive airway pressure. This therapy works by protruding and stiffening the tongue muscle thereby dilating the pharyngeal airway. However, only a small subset of patients with OSA have anatomy suitable for hypoglossal nerve stimulation therapy, as many patients continue to suffer from airway collapse even with stimulation of hypoglossal nerve musculature.

### SUMMARY

The subject technology is illustrated according to various aspects described below, including with reference to FIGS. 1-4D. Various examples of aspects of the subject technology are described as numbered clauses (1, 2, 3, etc.) for convenience. These are provided as examples and do not limit the subject technology.
1. An implantable device for treating sleep apnea, comprising: an electronics package; and
first and second elongate leads electrically coupled to the electronics package,
   wherein each of the first and second leads comprise:
   a first conductive region configured to be implanted at a first treatment site at an under-chin region of the patient's head, wherein the first conductive region is configured to deliver a first electrical signal to the first treatment site to thereby activate the genioglossus muscle, and
   a second conductive region configured to be implanted at a second treatment site at a neck region of the patient, wherein the second conductive region is configured to deliver a second electrical signal to the second treatment site to thereby activate an infrahyoid strap muscle.
2. The implantable device of Clause 1, wherein the electronics package is configured to be implanted at an under-chin region of the patient's head.
3. The implantable device of Clause 1 or Clause 2, wherein the first electrical signal is configured to activate the genioglossus muscle via electrical stimulation of the hypoglossal nerve.
4. The implantable device of any one of the previous Clauses, wherein the second electrical signal is configured to directly stimulate the infrahyoid strap muscle.
5. The implantable device of any one of the previous Clauses, wherein the second electrical signal is configured to stimulate the infrahyoid strap muscle via stimulation of the ansa cervicalis.
6. The implantable device of any one of the previous Clauses, wherein the second conductive region is distal of the first conductive region along a longitudinal axis of the respective first and second elongate leads.
7. The implantable device of any one of the previous Clauses, wherein the first and second elongate leads are configured to be implanted such that the first conductive regions are aligned with and extend along a left hypoglossal nerve and a right hypoglossal nerve, respectively.
8. The implantable device of any one of the previous Clauses, wherein the first and second elongate leads are configured to be implanted such that the second conductive regions are at or within a left sternothyroid muscle and a right sternothyroid muscle, respectively.
9. The implantable device of any one of the previous Clauses, wherein the second treatment site is on or within the sternothyroid muscle.
10. The implantable device of any one of the previous Clauses, further comprising an extension having a proximal end portion configured to be coupled to the electronics package and a distal end portion, and wherein proximal end portions of each of the first and second elongate leads are coupled to the distal end portion of the extension.
11. The implantable device of any one of Clauses 1-9, wherein each of the first and second elongate leads extends between a proximal end portion at the electronics package and a distal end portion.
12. The implantable device of any one of the previous Clauses, further comprising a first fixation element disposed at or near the first conductive region of the first elongate lead and a second fixation element disposed at or near the first conductive region of the second conductive lead, wherein each of the first and second fixation elements is configured to anchor the corresponding first or second elongate lead to tissue at the first treatment sites.
13. The implantable device of any one of the previous Clauses, further comprising a first fixation element disposed at or near the second conductive region of the first elongate lead and a second fixation element disposed at or near the second conductive region of the second conductive lead, wherein each of the first and second fixation elements is configured to anchor the corresponding first or second elongate lead to tissue at the second treatment sites.
14. An implantable device for treating sleep apnea, comprising:
an electronics package;
a first conductive region electrically coupled to the electronics package and configured to be implanted at a first treatment site at an under-chin region of the patient's head, wherein the first conductive region is configured to deliver a first electrical signal to the first treatment site to thereby activate the genioglossus muscle; and
a second conductive region electrically coupled to the electronics package and configured to be implanted at a second treatment site at a neck region of the patient, wherein the second conductive region is configured to deliver a second electrical signal to the second treatment site to thereby activate an infrahyoid strap muscle.
15. A method for treating sleep apnea, comprising:
positioning a first conductive element of an implantable lead on and/or in a sternothyroid muscle of a patient;
positioning a second conductive element of the lead proximate a hypoglossal nerve of the patient;
stimulating the sternothyroid muscle via the first conductive element; and
stimulating the hypoglossal nerve via the second conductive element to activate a genioglossus of the patient.
16. The method of Clause 15, wherein stimulating the sternothyroid muscle and stimulating the hypoglossal nerve occur at the same time.
17. The method of Clause 15, wherein the lead is a first lead, the sternothyroid muscle is a right sternothyroid muscle, and the hypoglossal nerve is a right hypoglossal nerve, and the method further comprises:
positioning a first conductive element of a second lead on and/or in a left sternothyroid muscle of the patient, wherein the first and second leads are electrically coupled to an implantable electronics package;
positioning a second conductive element of the second lead proximate a left hypoglossal nerve of the patient;
stimulating the left sternothyroid muscle via the first conductive element; and
stimulating the left hypoglossal nerve via the second conductive element to activate a genioglossus of the patient.
18. A method for treating sleep apnea, the method comprising:
implanting a neurostimulator comprising an electronics package and an elongate lead coupled to and extending from the electronics package, the elongate lead having a first stimulation zone and a second stimulation zone spaced apart along a longitudinal axis of the elongate lead, wherein implanting the neurostimulator comprises positioning the first stimulation zone on and/or in a sternothyroid muscle of a patient and positioning the second stimulation zone proximate a hypoglossal nerve of a patient;
delivering energy at the first stimulation zone to stimulate the sternothyroid muscle; and
delivering energy at the second stimulation zone to stimulate a genioglossus muscle of the patient.
19. A method for treating sleep apnea, comprising:
positioning a first conductive element of an implantable lead proximate a region of the ansa cervicalis that stimulates the sternothyroid;
positioning a second conductive element of the lead proximate a hypoglossal nerve of the patient;
stimulating the sternothyroid muscle via energy delivery through the first conductive element; and
stimulating the genioglossus muscle via energy delivery through the second conductive element.
20. The method of Clause 19, wherein stimulating the sternothyroid muscle and stimulating the genioglossus muscle occur at the same time.
21. A method for treating sleep apnea, comprising:
positioning a first conductive element of an implantable lead on and/or in a sternothyroid muscle of a patient;
positioning a second conductive element of the lead proximate a hypoglossal nerve of the patient;
depending on anatomy and/or position, stimulating one or both of the sternothyroid muscle via the first conductive element and the hypoglossal nerve via the second conductive element.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 schematically depicts selected nerves and muscles of the neck region of a patient.

FIG. 2 is a schematic illustration of a treatment system configured in accordance with several embodiments of the present technology.

FIG. 3 is a perspective view of a lead for use with the treatment devices of the present technology.

FIGS. 4A, 4B, 4C and 4D show a treatment device with the lead of FIG. 3 implanted in a human patient in accordance with several embodiments of the present technology.

### DETAILED DESCRIPTION

The present disclosure relates to systems and methods for improving SDB by activating one or more infrahyoid strap muscles. Non-limiting examples of SDBs are increased upper airway resistance including snoring; upper airway resistance syndrome (UARS); and sleep apnea. Sleep apnea can include OSA, central sleep apnea (CSA), and mixed sleep apnea. As used herein, “neuromodulation” or “neurostimulation” refers to exciting or inhibiting neural activity. Likewise, as used herein, “muscle stimulation” or “direct stimulation” refers to muscle activation via conductive elements placed in or on the targeted muscle. “Stimulation” alone refers to nerve stimulation, direct muscle stimulation, or both. A patient suffering from SDB includes a mammal, such as a human being.

The present disclosure provides methods and systems for treating SDB in a patient suffering therefrom by activating one or more infrahyoid strap muscles (e.g., the sternothyroid, the sternohyoid, the omohyoid, and the thyrohyoid). Activation of one or more infrahyoid muscles can be accomplished by stimulating an ansa cervicalis, including one or both of the superior root and the inferior root of the ansa cervicalis, alone or in combination with stimulating the HGN. Additionally or alternatively, activation of one or more infrahyoid muscles can be accomplished by directly stimulating one or more infrahyoid muscles, alone or in combination with stimulating the HGN. In either case, the stimulation can be electrical stimulation. Further, stimulation includes unilateral stimulation as well as bilateral stimulation of these nerve(s) and/or muscle(s). Without wishing to be bound by a particular mechanism of action, it is believed that activation of infrahyoid muscles (e.g., tightening of these muscles) can reduce upper airway compliance (e.g., stiffen the upper airway). Upper airway compliance can indicate the potential of the airway to collapse and can be relevant to treating SDB. As explained below, the infrahyoid muscles include the sternohyoid muscle, the sternothyroid muscle, the omohyoid muscle, and the thyrohyoid muscle. The present disclosure provides a method of activating one or more of these muscles either alone or in combination with activating the genioglossus muscle. Activation of the genioglossus muscle can be accomplished by neuromodulating the hypoglossal nerve (HGN).

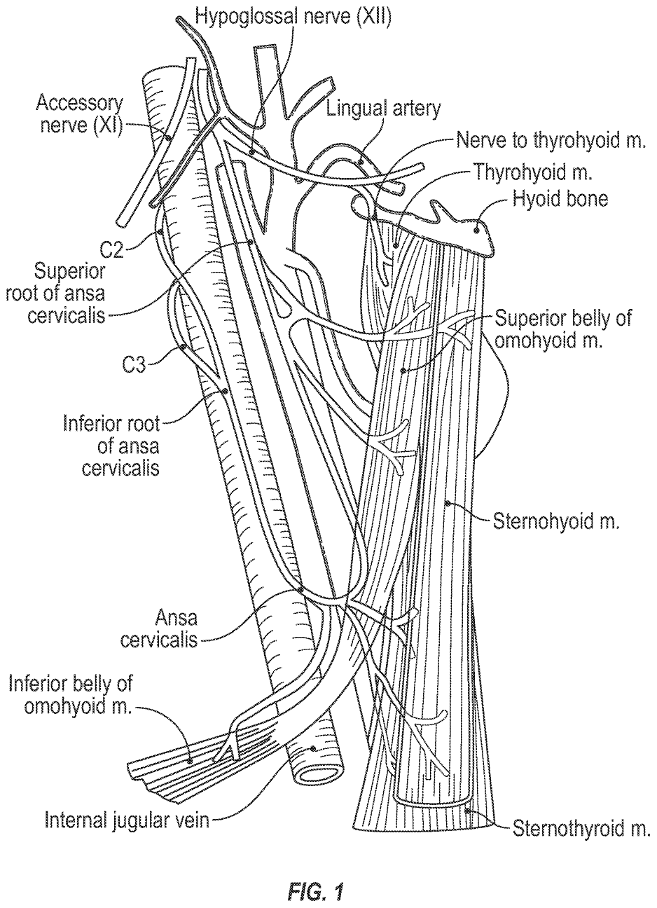

With reference to FIG. 1, in some embodiments the treatment device is configured to activate one or more infrahyoid strap muscles via stimulation of the ansa cervicalis. The infrahyoid strap muscles can be variably innervated by nerve fiber contributions from both the superior and inferior roots of the ansa cervicalis. It should be noted that FIG. 1 illustrates only a few branching patterns of the ansa cervicalis and that actual anatomic branching patterns can be different from patient to patient and likely include more branches. Normal anatomic variants may necessitate use of one or more different target sites in different patients to achieve desired stimulation of a targeted infrahyoid strap muscle. In certain aspects and with reference to FIG. 1, a neuromodulation signal can be delivered to a target site proximate to the ansa cervicalis that also innervates the superior belly of the sternohyoid muscle and/or inferior belly of the sternohyoid muscle to activate part of or all of the sternohyoid muscle. For example, a target site can be proximal to or at the branch point of the superior root of the ansa cervicalis innervating the sternohyoid muscle such that the sternohyoid muscle is activated as well as the sternothyroid muscle. In certain aspects, delivering a neuromodulation signal proximal to or at the branch point of the superior root of the ansa cervicalis can also activate part or all of the omohyoid muscle. If the target site were distal to the superior root of the ansa cervicalis but proximal of a subsequent branch point, a neuromodulation signal may only activate the sternohyoid muscle and/or omohyoid muscle and not necessarily the sternothyroid muscle. Without wishing to be bound by a particular mechanism of action, it is believed that activation of at least the sternothyroid muscle can stiffen the patient's upper airway thereby improving the patient's SDB.

In certain aspects, a neuromodulation signal is delivered to a target site along the inferior root of the ansa cervicalis, thereby innervating the sternothyroid muscle, sternohyoid muscle, and omohyoid muscle. In certain aspects, a neuromodulation signal can be delivered simultaneously to target sites along the inferior and superior roots of the ansa cervicalis in order to stimulate nerve branches along both the superior and inferior roots. In certain aspects, delivering a neuromodulation signal to a target site proximate to or at the branch point of the common trunk nerve or nerves arising from the loop of the ansa cervicalis combining nerve fibers from the superior root and inferior root and supplying at least the sternothyroid muscle and variably the sternohyoid muscle and omohyoid muscle can activate at least the sternothyroid muscle and in certain aspects, the sternohyoid muscle and in certain aspects the omohyoid muscle. In certain aspects, delivering a neuromodulation signal to a target site proximate to or at the branch point of the sternothyroid muscle nerve or nerves from the common trunk can activate the sternothyroid muscle. The branches to the sternothyroid muscle can be a single nerve fiber or several closely located nerve fibers traveling together. In some embodiments, a conductive region of the treatment device can be placed proximal or distal to the branch of the ansa cervicalis innervating the omohyoid muscle and/or branch innervating the sternohyoid muscle such that stimulation via the conductive region only captures the sternothyroid fibers, or only the sternothyroid and sternohyoid fibers (and selectively avoids the omohyoid fibers). In certain aspects, a cuff electrode or electrodes could surround a single fiber or multiple fibers innervating the sternothyroid muscle, the sternohyoid muscle, and/or the omohyoid muscle. In some embodiments, a plurality of band electrodes can be positioned on or be aligned with multiple branches of the ansa cervicalis innervating the sternothyroid muscle, the sternohyoid muscle, and/or the omohyoid muscle. It should be noted that the above target sites are only exemplary and the treatment device can be placed at other parts of the ansa cervicalis including branches thereof.

Delivering a neuromodulation signal, such as an electrical neuromodulation signal, can be accomplished by implanting one or more electrical leads. To deliver the neuromodulation signal, one or more conductive regions of an implantable treatment device can be positioned proximate to a target site innervating one or more infrahyoid strap muscles. The conductive region(s) can be placed proximate to a target site in a variety of different ways, such as, for example, transcutaneously, percutaneously, subcutaneously, intramuscularly, intraluminally, transvascularly, intravascularly, or via direct open surgical implantation. The conductive regions can also have different form factors such as, for example, one or more band electrodes positioned on an elongated lead that is electrically coupled to an implantable stimulator, one or more nerve cuff electrodes that are electrically coupled to an implantable stimulator, or an injectable microstimulator.

An electrode or neurostimulator can be placed on the same or different target sites. For example, if the target sites include the superior root of the ansa cervicalis and the inferior root of the ansa cervicalis, a separate lead (with one or more band electrodes) or nerve cuff electrode can be placed on each root with each lead or nerve cuff electrode having its own cathode and anode but connected to the same pulse generator or separate leads or nerve cuff electrodes connected to the same pulse generator but one lead or nerve cuff electrode serves as the cathode and the other serves as the anode, where the electrical field generated captures both roots. A single lead or nerve cuff electrode can be positioned on a right ansa cervicalis while multiple leads or nerve cuff electrodes can be positioned on a left ansa cervicalis, or vice versa. Likewise, a single lead or nerve cuff electrode can be positioned on a right ansa cervicalis and a single lead or nerve cuff electrode can be positioned on a left ansa cervicalis. In some embodiments, multiple leads or nerve cuff electrodes are positioned on a right ansa cervicalis and multiple leads or nerve cuff electrodes are positioned on a left ansa cervicalis. In certain aspects, a single or multiple leads or nerve cuff electrodes are positioned only on a right ansa cervicalis or a left ansa cervicalis. When one or more conductive regions are positioned on both the right and left sides, the branches targeted on each side and/or the muscles targeted on each side may be the same or different.

Still with reference to FIG. 1, in some embodiments the treatment device is configured to activate one or more infrahyoid strap muscles directly, either in combination with or instead of activation via the ansa cervicalis. In some embodiments, the lead can include an intramuscular electrode and one or more anchors. The lead can be tunneled subcutaneously within a sheath to a target infrahyoid strap muscle, and the sheath can be retracted to deploy the anchors and bury the intramuscular electrode in the muscle. When implanted, the intramuscular electrode is in direct contact with the muscle but separated by muscle tissue from the innervating motor nerves.

According to several embodiments, the HGN can be stimulated to activate the genioglossus muscle and/or geniohyoid muscle. This stimulation can be in combination with stimulation of the ansa cervicalis, direct stimulation of the infrahyoid strap muscles, or both. Within a given treatment program, stimulation of the HGN can occur at the same time or different times as stimulation of the ansa cervicalis and/or direct stimulation of the infrahyoid strap muscles. Without wishing to be bound by a particular mechanism of action, it is believed that activation of one or more infrahyoid strap muscles, such as the sternothyroid muscle, can stiffen the patient's airway and activation of the genioglossus muscle can cause the tongue to move forward and dilate/reinforce the patient's upper airway thereby improving a patient's SDB.

According to several embodiments, the electrodes can be positioned at a location that is close to the region of the muscle where the major portion of the motor nerve fibers are located, e.g., the motor end plate. The motor end plate, or motor point, can be identified by moving a stimulating electrode across the muscle to locate the position that requires the least amount of stimulation to activate (fully) the muscle. When using surface or intramuscular electrodes, the electrodes may be “close” to the motor point, but not directly in contact with the nerves. Accordingly, both muscle stimulation and motor end plate stimulation is achieved. The electrodes may be positioned on the surface of the muscle or within the muscle, have larger electrodes compared to nerve stimulation electrodes and larger pulse widths. According to several of such methods, stimulating the muscle also captures the motor end plate. There may be more than one motor point, based on the nerve branching and overall insertion points.

In some aspects of the technology, surface electrodes can be positioned on the muscle belly, where the electrodes are spaced appropriately, starting from a caudal location to a cranial location on the surface of the muscle. The electrodes can be tested during and after implantation to determine the best location for stabilizing the lower airway, which may be different than the location of best activation threshold.

### I. Treatment Systems

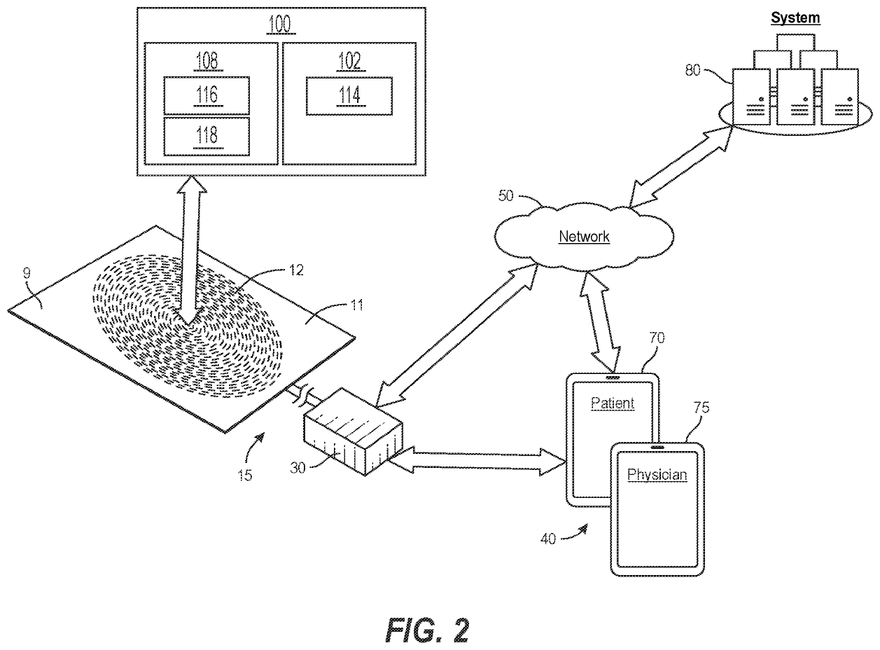

Aspects of the present disclosure provide devices and systems for improving SDB in a patient suffering therefrom. FIG. 2 shows a treatment system 10 for treating SDB configured in accordance with the present technology. The system 10 can include an implantable treatment device 100 and an external system 15 configured to wirelessly couple to the treatment device 100. The treatment device 100 can include a lead 102 having a plurality of conductive elements 114 and an electronics package 108 having a first antenna 116 and an electronics component 118. The treatment device 100 is configured to be implanted at treatment sites in the patient's head and neck to activate both the genioglossus muscle and one or more of the infrahyoid strap muscles.

In use, the electronics package 108 or one or more elements thereof can be configured provide a stimulation energy to the conductive elements 114 that has a pulse width, amplitude, duration, frequency, duty cycle, and/or polarity such that the conductive elements 114 apply an electric field at the treatment site that 1) modulates the hypoglossal nerve, 2) modulates the ansa cervicalis, and/or 3) directly stimulates an infrahyoid strap muscle. The stimulation energy can be delivered according to a periodic waveform including, for example, a charge-balanced square wave comprising alternating anodic and cathodic pulses.

The pulse width, amplitude, duration, frequency, duty cycle, polarity, and/or waveform may be different for different conductive elements 114, depending on the targeted anatomy.

One or more pulses of the neurostimulation energy for stimulating the HGN or ansa cervicalis can have a pulse width between about 10 μs and about 1000 μs, between about 50 μs and about 950 μs, between about 100 μs and about 900 μs, between about 150 μs and about 800 μs, between about 200 μs and about 850 μs, between about 250 μs and about 800 μs, between about 300 μs and about 750 μs, between about 350 μs and about 700 μs, between about 400 μs and about 650 μs, between about 450 μs and about 600 μs, between about 500 μs and about 550 μs, about 50 μs, about 100 μs, about 150 μs, about 200 μs, about 250 μs, about 300 μs, about 350 μs, about 400 μs, about 450 μs, about 500 μs, about 550 μs, about 600 μs, about 650 μs, about 700 μs, about 750 μs, about 800 μs, about 850 μs, about 900 μs, about 950 μs, and/or about 1000 μs. In some embodiments, one or more pulses of the stimulation energy has a pulse width of between about 50 μs and about 450 μs.

The pulse width of the stimulation energy for directly stimulating the infrahyoid strap muscles can be between about 150 μs and about 2 ms, between about 250 μs and about 2 ms, greater than about 250 μs, greater than about 300 μs, greater than about 350 μs, greater than about 400 μs, greater than about 500 μs, greater than about 600 μs, greater than about 700 μs, greater than about 800 μs, greater than about 900 μs, greater than about 1 ms, greater than about 1.1 ms, greater than about 1.2 ms, greater than about 1.3 ms, greater than about 1.4 ms, greater than about 1.5 ms, greater than about 1.6 ms, greater than about 1.7 ms, greater than about 1.8 ms, greater than about 1.9 ms, or greater than about 2 ms. In some cases, the pulse width of the stimulation energy for directly stimulating the infrahyoid strap muscles can be greater than the pulse width for stimulating the HGN or ansa cervicalis. It may be advantageous to use a greater pulse width for direct muscle activation as the distribution of motor points along the muscle can be unpredictable. Increasing the pulse width injects stimulation energy to activate a greater number of end plates.

One or more pulses of the neurostimulation energy for stimulating the HGN, ansa cervicalis, or strap muscle directly can have an amplitude sufficient to cause an increase in phasic activity of a desired muscle. For example, one or more pulses of the stimulation energy can have a current-controlled amplitude between about 0.1 mA and about 5 mA. In some embodiments, the stimulation energy has an amplitude of about 0.3 mA, about 0.4 mA, about 0.5 mA, about 0.6 mA, about 0.7 mA, about 0.8 mA, about 0.9 mA, about 1 mA, about 1.5 mA, about 2 mA, about 2.5 mA, about 3 mA, about 3.5 mA, about 4 mA, about 4.5 mA, and/or about 5 mA. Additionally or alternatively, an amplitude of one or more pulses of the stimulation energy can be voltage-controlled. An amplitude of one or more pulses of the stimulation energy can be based at least in part on a size and/or configuration of the conductive elements 114, a location of the conductive elements 114 in the patient, etc.

In comparison to nerve action potentials, muscles have a longer recharge phase. The direct muscle stimulation energy may have the same charge balance as the neurostimulation energy, and potentially the same stimulation phase, but a longer recharge phase with a smaller amplitude. A longer recharge phase can also be beneficial for direct muscle activation because of the potentially greater conductive surface area being used for the stimulation. Muscle activation is much slower than nerve activation, and typically it is better to avoid reactivating the muscle on the recharge phase. When stimulating both muscle tissue and potentially the motor nerve end plate, asymmetrical pulses, which have a longer anodic phase, are believed by the inventor(s) to have lower thresholds compared to symmetrical waveforms. Likewise, in such cases, the inventor(s) believe that longer pulse widths may be beneficial for reducing the amplitude thresholds for activation. Accordingly, longer pulse widths and/or asymmetrical waveforms with longer anodic pulses may reduce the activation thresholds for both surface or intramuscular electrodes. Such a combination of parameters may beneficially stimulate muscle, motor end plate(s), or both.

A frequency of the pulses for stimulating the HGN, ansa cervicalis, or strap muscle directly can be between about 10 Hz and about 150 Hz, between about 10 Hz and about 100 Hz, between about 20 Hz and about 40 Hz, about 10 Hz, about 15 Hz, about 20 Hz, about 25 Hz, about 30 Hz, about 35 Hz, about 40 Hz, about 45 Hz, and/or about 50 Hz. In some embodiments, the frequency can be based on a desired effect of the stimulation energy on one or more muscles or nerves. For example, lower frequencies may induce a muscular twitch whereas higher frequencies may include complete contraction of a muscle.

The external system 15 can comprise an external device 11 and a control unit 30 communicatively coupled to the external device 11. In some embodiments, the external device 11 is configured to be positioned proximate a patient's head while they sleep. The external device 11 can comprise a carrier 9 integrated with a second antenna 12. Additional details regarding the external system 15 and the external device 11 can be found in U.S. Patent Application No. 63/483,961, filed Feb. 8, 2023, which is incorporated by reference herein in its entirety. While the control unit 30 is shown separate from the external device 11 in FIG. 2, in some embodiments the control unit 30 can be integrated with and/or a portion of the external device 11. The second antenna 12 can be configured for multiple purposes. For example, the second antenna 12 can be configured to power the treatment device 100 through electromagnetic induction. Electrical current can be induced in the first antenna 116 when it is positioned above the second antenna 12 of the external device 11, in an electromagnetic field produced by second antenna 12. The first and second antennas 116, 12 can also be configured transmit data to and/or receive data from one another via one or more wireless communication techniques (e.g., Bluetooth, WiFi, USB, etc.) to facilitate communication between the treatment device 100 and the external system 15. This communication can, for example, include programming, e.g., uploading software/firmware revisions to the treatment device 100, changing/adjusting stimulation settings and/or parameters, and/or adjusting parameters of control algorithms.

The control unit 30 of the external system 15 can include a processor and/or a memory that stores instructions (e.g., in the form of software, code or program instructions executable by the processor or controller) for causing the external device to generate an electromagnetic field according to certain parameters provided by the instructions. The external system can include and/or be configured to be coupled to a power source such as a direct current (DC) power supply, an alternating current (AC) power supply, and/or a power supply switchable between DC and AC. The processor of the external system can be used to control various parameters of the energy output by the power source, such as intensity, amplitude, duration, frequency, duty cycle, and polarity. Instead of or in addition to a processor, the external system can include drive circuitry. In such embodiments, the external system can include hardwired circuit elements to provide the desired waveform delivery rather than a software-based generator. The drive circuitry can include, for example, analog circuit elements (e.g., resistors, diodes, switches, etc.) that are configured to cause the power source to supply energy to the second antenna 12 to produce an electromagnetic field according to the desired parameters. In some embodiments, the treatment device 100 can be configured for communication with the external system via inductive coupling.

The system 10 can also include a user interface 40 in the form of a patient device 70 and/or a physician device 75. The user interface(s) 40 can be configured to transmit and/or receive data with the external system 15, the second antenna 12, the control unit 30, the treatment device 100, and/or the remote computing device(s) 80 via wired and/or wireless communication techniques (e.g., Bluetooth, WiFi, USB, etc.). In the example configuration of FIG. 2, both the patient device 70 and physician device 75 are smartphones. The type of device could, however, vary. One or both of the patient device 70 and physician device 75 can have an application or “app” installed thereon that is user specific, e.g., a patient app or a physician app, respectively. The patient app can allow the patient to execute certain commands necessary for controlling operation of treatment device 100, such as, for example, start/stop therapy, increase/decrease stimulation power or intensity, and/or select a stimulation program. In addition to the controls afforded the patient, the physician app can allow the physician to modify stimulation settings, such as pulse settings (patterns, duration, waveforms, etc.), stimulation frequency, amplitude settings, and electrode configurations, closed loop and open loop control settings and tuning parameters for the embedded software that controls therapy delivery during use.

The patient and/or physician devices 70, 75 can be configured to communicate with the other components of the system 10 via a network 50. The network 50 can be or include one or more communications networks, such as any of the following: a wired network, a wireless network, a metropolitan area network (MAN), a local area network (LAN), a wide area network (WAN), a virtual local area network (VLAN), an internet, an extranet, an intranet, and/or any other suitable type of network or combinations thereof. The patient and/or physician devices 70, 75 can be configured to communicate with one or more remote computing devices 80 via the network 50 to enable the transfer of data between the devices 70, 75 and the remote computing device(s) 80. Additionally, the external system 15 can be configured to communicate with the other components of the system 10 via the network 50. This can also enable the transfer of data between the external system 15 and remote computing device(s) 80.

The external system 15 can receive the programming, software/firmware, and settings/parameters through any of the communication paths described above, e.g., from the user interface(s) 40 directly (wired or wirelessly) and/or through the network 50. The communication paths can also be used to download data from the treatment device 100, such as measured data regarding completed stimulation therapy sessions, to the external system 15. The external system 15 can transmit the downloaded data to the user interface 40, which can send/upload the data to the remote computing device(s) 80 via the network 50.

In addition to facilitating local control of the system 10, e.g., the external system 15 and the treatment device 100, the various communication paths shown in FIG. 2 can also enable:
Distributing from the remoting computing device(s) 80 software/firmware updates for the patient device 70, physician device 75, external system 15, and/or treatment device 100.
Downloading from the remote computing device(s) 80 therapy settings/parameters to be implemented by the patient device 70, physician device 75, external system 15, and/or treatment device 100.
Facilitating therapy setting/parameter adjustments/algorithm adjustments by a remotely located physician.
Uploading data recorded during therapy sessions.
Maintaining coherency in the settings/parameters by distributing changes and adjustments throughout the system components.

The therapeutic approach implemented with the system 10 can involve implanting only the treatment device 100 and leaving the external system 15 as an external component to be used only during the application of therapy. To facilitate this, the treatment device 100 can be configured to be powered by the external system 15 through electromagnetic induction. In operation, the second antenna 12, operated by control unit 30, can be positioned external to the patient in the vicinity of the treatment device 100 such that the second antenna 12 is close to the first antenna 116 of the treatment device 100. In some embodiments, the second antenna 12 is carried by a flexible carrier 9 that is configured to be positioned on or sufficiently near the sleeping surface while the patient sleeps to maintain the position of the first antenna 116 within the target volume of the electromagnetic field generated by the second antenna 12. Through this approach, the system 10 can deliver therapy to improve SDB (such as OSA), for example, by stimulating the HGN, ansa cervicalis, and/orinfrahyoid strap muscles through a shorter, less invasive procedure. The elimination of an on-board, implanted power source in favor of an inductive power scheme can eliminate the need for batteries and the associated battery changes over the patient's life.

In some embodiments, the system 10 can include one or more sensors (not shown), which may be implanted and/or external. For example, the system 10 can include one or more sensors carried by (and implanted with) the treatment device 100. Such sensors can be disposed at any location along the lead 102 and/or electronics package 108. In some embodiments, one, some, or all of the conductive elements 114 can be used for both sensing and stimulation. Use of a single structure or element as the sensor and the stimulating electrode reduces the invasive nature of the surgical procedure associated with implanting the system, while also reducing the number of foreign bodies introduced into a subject. In certain embodiments, at least one of the conductive elements 114 is dedicated to sensing only.

In addition to or instead of inclusion of one or more sensors on the treatment device 100, the system 10 can include one or more sensors separate from the treatment device 100. In some embodiments, one or more of such sensors are wired to the treatment device 100 but implanted at a different location than the treatment device 100. In some embodiments, the system 10 includes one or more sensors that are configured to be wirelessly coupled to the treatment device 100 and/or an external computing device (e.g., control unit 30, user interface 40, etc.). Such sensors can be implanted at the same or different location as the treatment device 100, or may be disposed on the patient's skin.

The one or more sensors can be configured to record and/or detect physiological data (e.g., data originating from the patient's body) over time including changes therein. The physiological data can be used to select certain stimulation parameters and/or adjust one or more stimulation parameters during therapy. Physiological data can include an electromyography (EMG) signal, temperature, movement, audio data, heart rate, pulse oximetry, eye motion, and/or combinations thereof. In some embodiments, the physiological data can be used to detect and/or anticipate other physiological parameters. For example, the one or more sensors can be configured to sense an EMG signal which can be used to detect and/or anticipate physiological data such as phasic contraction of anterior lingual musculature (such as phasic genioglossus muscle contraction) and underlying tonic activity of anterior lingual musculature (such as tonic activity of the genioglossus muscle). Phasic contraction of the genioglossus muscle can be indicative of inspiration, particularly the phasic activity that is layered within the underlying tonic tone of the genioglossus muscle. Changes in physiological data include changes in one or more parameters of a measured signal (e.g., frequency, amplitude, spike rate, etc.), changes in phasic contraction of anterior lingual musculature (such as phasic genioglossus muscle contraction), changes in underlying tonic activity of anterior lingual musculature (such as changes in tonic activity of the genioglossus muscle), and combinations thereof. In particular, changes in phasic contraction of the genioglossus muscle can indicate a respiration or inspiration change and can be used to trigger stimulation. Such physiological data and changes therein can be identified in recorded EMG signals, such as during different phases of respiration including inspiration. As such, the one or more sensors can include EMG sensors. The one or more sensors can also include, for example, wireless or tethered sensors that measure, body temperature, movement (e.g., an accelerometer), breath sounds (e.g., audio sensors), heart rate, pulse oximetry, eye motion, etc. In addition to the genioglossus muscle, the sensors can be configured to record and/or detect physiological data characterizing one or more of the infrahyoid strap muscles.

In operation, the physiological data provided by the one or more sensors enables closed-loop operation of the treatment device 100. For example, the sensed EMG responses from the genioglossus muscle and/or one or more infrahyoid strap muscles can enable closed-loop operation of the treatment device 100 while eliminating the need for a chest lead to sense respiration. Operating in closed-loop, the treatment device 100 can maintain stimulation synchronized with respiration, for example, while preserving the ability to detect and account for momentary obstruction. The treatment device 100 can also detect and respond to snoring, for example.

The system 10 can be configured to provide open-loop control and/or closed-loop stimulation to configure parameters for stimulation. In other words, with respect to closed-loop stimulation, the system 10 can be configured to track the patient's respiration (such as each breath of the patient) and stimulation can be applied during inspiration, for example. However, with respect to open-loop stimulation, stimulation can be applying without tracking specific physiological data, such as respiration or inspiration. However, even under such an “open loop” scenario, the system 10 can still adjust stimulation and record data, to act on such information. For example, one way the system 10 can act upon such information is that the system 10 can configure parameters for stimulation to apply stimulation in an open loop fashion but can monitor the patient's respiration to know when to revert to applying stimulation on a breath to breath, close-loop fashion such that the system 10 is always working in a close looped algorithm to assess data. Treatment parameters of the system may be automatically adjusted in response to the physiological data. The physiological data can be stored over time and examined to change the treatment parameters; for example, the treatment data can be examined in real time to make a real time change to the treatment parameters.

Operating in real-time, the treatment device 100 can record data (e.g., via one or more sensors) related to the stimulation session including, for example, stimulation settings, EMG responses, respiration, sleep state including different stages of REM and non-REM sleep, etc. For example, changes in phasic and tonic EMG activity of the genioglossus muscle during inspiration can serve as a trigger for stimulation or changes in stimulation can be made based on changes in phasic and tonic EMG activity of the genioglossus muscle during inspiration or during different sleep stages. After the sleep session, this recorded data can be uploaded to the user interface 40 and to the remote computing device(s) 80. Also, the patient can be queried to use the interface 40 to log data regarding their perceived quality of sleep, which can also be uploaded to the remote computing device(s) 80. Offline, the remote computing device(s) 80 can execute a software application to evaluate the recorded data to determine whether settings and control parameters can be adjusted to further optimize the stimulation therapy. The software application can, for example, include artificial intelligence (AI) models that, learn from recorded therapy sessions, how certain adjustments affect the therapeutic outcome for the patient. In this manner, through AI learning, the model can provide patient-specific optimized therapy.

### II. Neuromodulation Devices

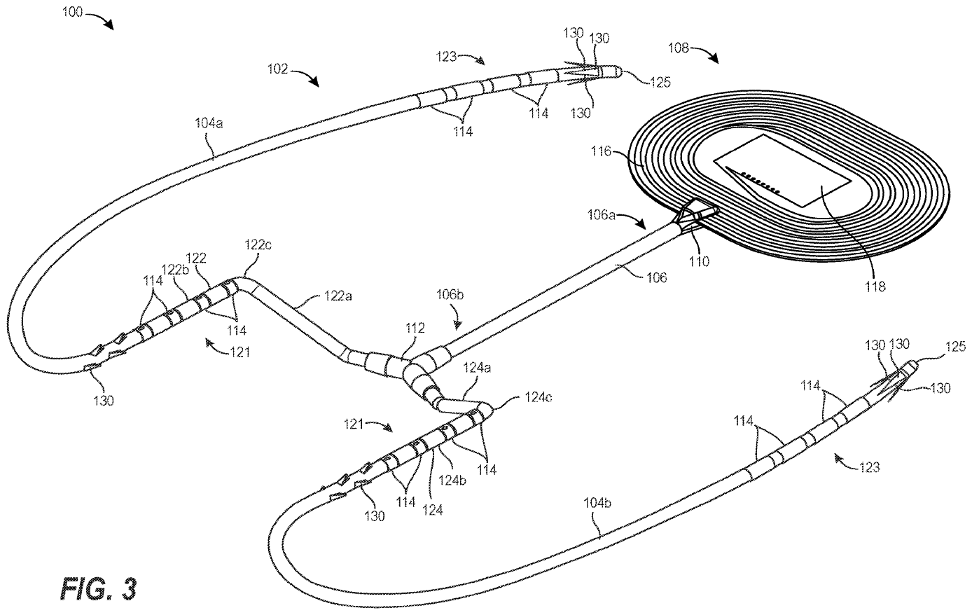

FIG. 3 shows an example treatment device 100 configured in accordance with several embodiments of the present technology. As previously mentioned, the device 100 can be configured to be implanted in the patient's head and neck region to deliver electrical energy at the treatment site to activate one or more tongue protruser muscles (e.g., the genioglossus, the geniohyoid, etc.) and one or more infrahyoid strap muscles (e.g., the sternothyroid muscle, the sternohyoid muscle, the omohyoid muscle, and the thyrohyoid muscle). The device 100 can include an electronics package 108 and a lead 102 coupled to and extending away from the electronics package 108. The lead 102 can comprise first and second elongate arms 104a, 104b (collectively, “elongate arms 104”) and an extension portion 106 extending between the elongate arms 104 and the electronics package 108. The extension portion 106 can have a proximal end portion 106a coupled to the electronics package 108 via a first connector 110 and a distal end portion 106b coupled to the elongate arms 104 via a second connector 112. In some embodiments, the lead 102 does not include an extension portion 106 and the proximal ends of the elongate arms 104 are coupled directly to the electronics package 108. In other words, the lead 102 need not be a single continuous member but may comprise multiple arms or protrusions extending separately from the electronics package 108. Moreover, the lead 102 can include more than two elongate arms, such as three elongate arms, four elongate arms, five elongate arms, six elongate arms, etc.

The treatment device 100 can include a plurality of treatment zones comprising one or more conductive elements 114. At least one of the treatment zones is disposed along the lead 102 such that, when the treatment device 100 is implanted, the treatment zone is configured to be positioned at an under-chin region of the patient to stimulate the HGN to activate the genioglossus. As used herein, “under-chin region” refers to an anatomical region bound anteriorly and laterally by the patient's mandible, superiorly by the superior surface of the tongue, and inferiorly by the patient's platysma. Such an anatomical region can include, for example, a submental region and a sublingual region. At least another one of the treatment zones is disposed along the lead 102 such that, when the treatment device 100 is implanted, the treatment zone is configured to be positioned at a neck region of the patient to stimulate the ansa cervicalis (to activate one or more infrahyoid muscles) and/or directly stimulate one or more infrahyoid strap muscles.

As shown in FIG. 3, in some embodiments the treatment device 100 can comprise multiple treatment zones on a single elongate arm 104. For example, the first elongate arm 104a can comprise a first treatment zone 121 configured to be positioned at an under-chin region of the patient to stimulate the HGN to activate the genioglossus, and a second treatment zone 123 configured to be positioned at a neck region of the patient to stimulate the ansa cervicalis (to activate one or more infrahyoid muscles) and/or directly stimulate one or more infrahyoid strap muscles. Likewise, the second elongate arm 104b can comprise a first treatment zone 121 configured to be positioned at an under-chin region of the patient to stimulate the HGN to activate the genioglossus, and a second treatment zone 123 configured to be positioned at a neck region of the patient to stimulate the ansa cervicalis (to activate one or more infrahyoid muscles) and/or directly stimulate one or more infrahyoid strap muscles. The treatment zones 121, 123 can be spaced apart along the longitudinal axis of the respective elongate arm 104 such that the first zone 121 is proximal of the second zone 123 and spaced apart by a non-conductive region.

The elongate arms 104a, 104b can each extend distally and laterally from the second connector 112 and/or the distal end portion 106b of the extension portion 106. Additional details regarding the geometry and construction of the elongate arms 104 can be found in U.S. Application No. 63/377,969, filed Sep. 30, 2022, which is incorporated by reference herein in its entirety. In some embodiments, one or both elongate arms 104 have more or fewer than two treatment zones, and different arms can have the same number or a different number of treatment zones, and the same or different placement of treatment zones along the respective arms 104.

The first and second treatment zones 121, 123 can each comprise a plurality of conductive elements 114 configured to be electrically coupled to the electronics package 108 and configured to deliver and/or receive electrical energy. As shown in FIG. 3, in some embodiments the first treatment zone 121 includes a plurality of first conductive elements 114 and the second treatment zone 123 includes a plurality of second conductive elements 114.

The type, number, and arrangement of conductive elements 114 within a given treatment zone can be tailored to the treatment requirements of the specific zone. The choice of conductive materials and geometric design can be determined by the intended location on the nerve or muscle, and ability to satisfy the requirements of material compatibility, mechanical compatibility and the ability to transfer the required electrical charges without tissue or material deterioration. It may be advantageous, for example, for the conductive elements 114 in the first treatment zone 121 to be band electrodes or cuff electrodes for nerve stimulation while the conductive elements 114 in the second treatment zone 123 to be configured to directly stimulate the muscle. For example, the second treatment zones 123 can comprise one or more intramuscular electrodes. In some cases, it may be beneficial for the conductive elements 114 within the second treatment zones 123 to have a greater total surface area (relative to the conductive elements 114 in the first treatment zones 121) and thus generate a larger electric field. In some embodiments, the conductive element(s) 114 within the second treatment zones 123 can comprise one or more wires coiled into a helix such bending forces on the conductive element transform into a torsional force. Such a helical configuration can be beneficial to improving the longevity of the intramuscular conductive elements 114. The conductive element(s) 114 in the second treatment zones 123 can have a strength, flexibility and tolerance of mechanical deformation sufficient for intramuscular placement.

Different treatment zones, whether on the same elongate arm or different elongate arms, can have the same or different number of conductive elements 114, the same or different arrangement of conductive elements 114, and the same or different type of conductive elements 114.

In some embodiments, the first and second treatment zones 121, 123 may also comprise one or more fixation elements 130. The fixation elements 130 can be configured to securely, and optionally releasably, engage patient tissue to prevent or limit movement of the lead body 104 relative to the tissue. The fixation elements 130 can be disposed at one or both sides of the conductive elements 114 within a given treatment zone and/or between two or more of the conductive elements 130 within a given treatment zone. Additionally or alternatively, the treatment device 100 can include fixation elements 130 at other locations along the lead 102 and/or elongate arms 104, such as in between the treatment zones 121, 123 along the longitudinal axis of the respective arm 104, proximate or on one or both of the first and second connectors 110, and/or along the extension portion 106. Different treatment zones, whether on the same elongate arm or different elongate arms, can have the same or different number of fixation elements 130, the same or different arrangement of fixation elements 130, and the same or different type of fixation elements 130. The type, number, and arrangement of fixation elements 130 can be tailored to each treatment zone to advantageously address different energy delivery requirements and local tissue.

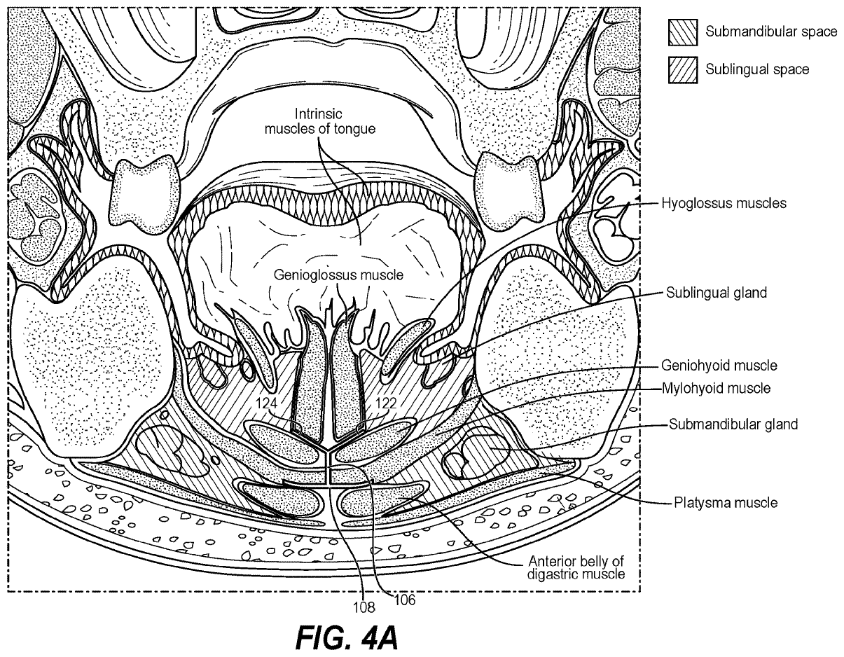

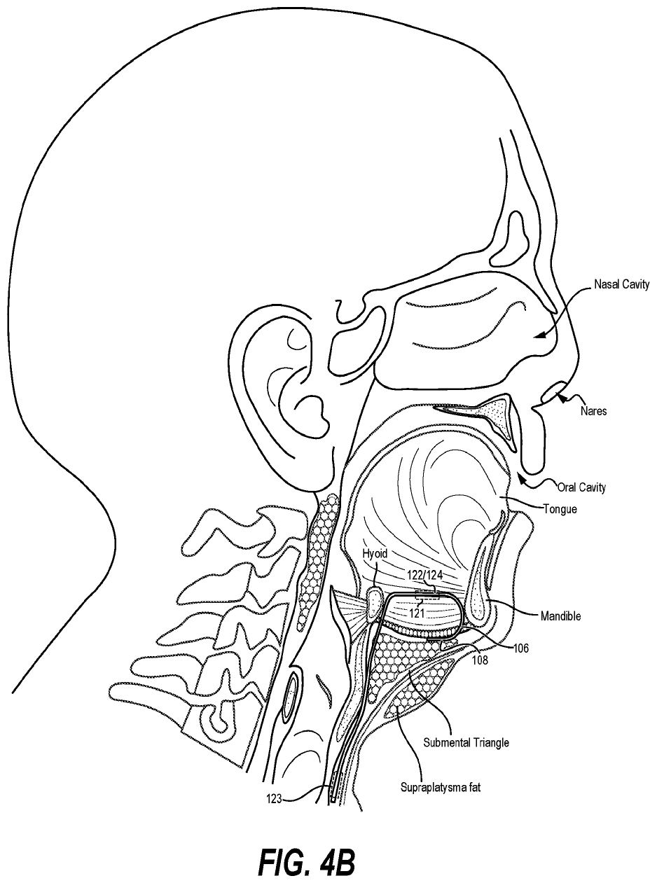

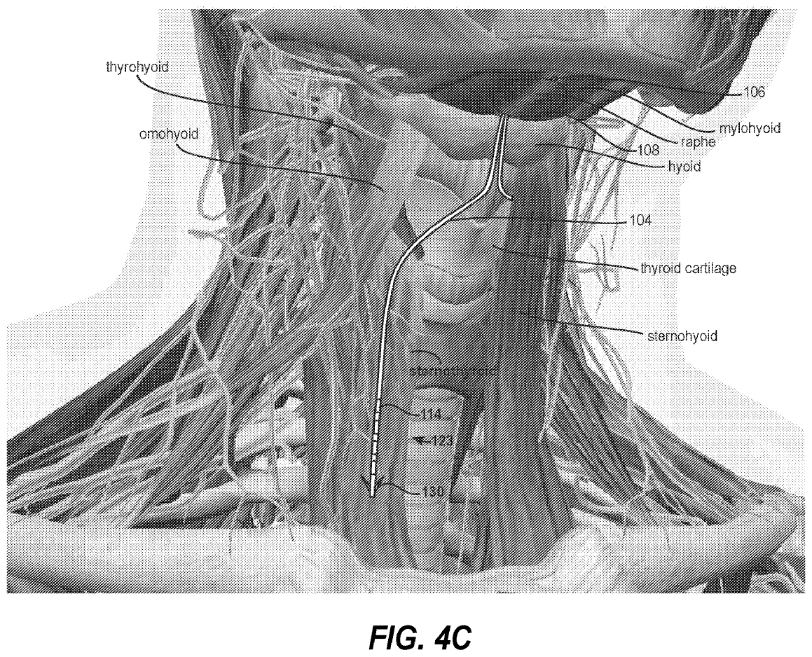

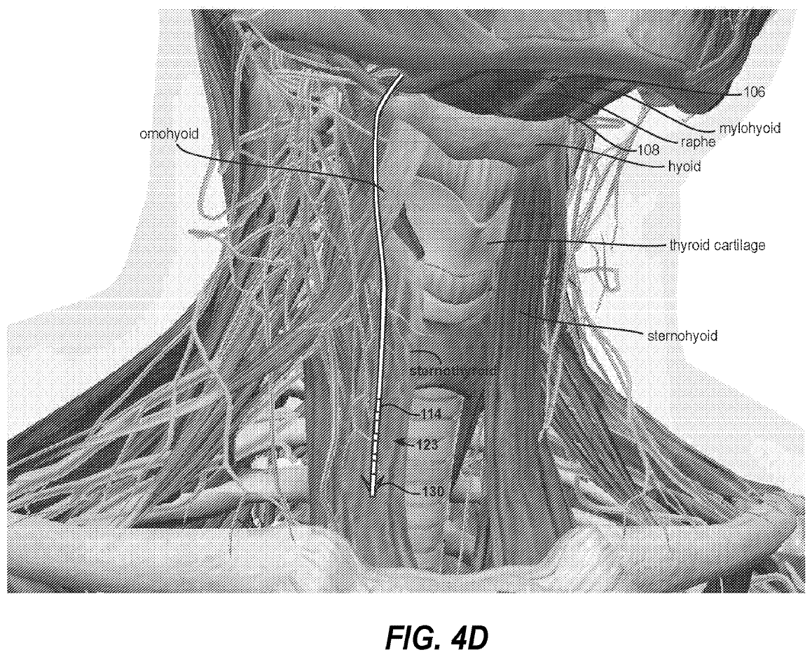

The electronics package 108 can be configured to supply electrical current to the conductive elements 114 (e.g., to stimulate) and/or receive electrical energy from the conductive elements 114 (e.g., to sense physiological data). The extension portion 106 of the lead 102 can mechanically and/or electrically couple the electronics package 108 to the elongate arms 104. The extension portion 106 can comprise a polymeric material such as, but not limited to, a thermoplastic elastomer, a thermoplastic polyurethane, a silicone, or other suitable materials. The extension portion 106 can be sufficiently flexible such that it can bend so as to position at least a proximal portion of the elongate arms 104 on top of, but spaced apart from, the electronics package 108. As discussed in greater detail below with reference to FIGS. 4A-4D, a portion of the treatment device 100 is configured to be implanted within both a submental region and a sublingual region such that the electronics package 108 and first treatment zones 121 are vertically stacked with one or more muscle and/or other tissue layers positioned therebetween. The flexibility and length of the extension portion 106 enables such a configuration. The elongate arms 124 may be configured to extend inferiorly along the patient's neck, which can be enabled by the flexibility and length of the elongate arms 104.

In some embodiments, the extension portion 106 comprises a sidewall defining a lumen extending through the extension portion 106. The conductive elements 114 can be electrically coupled to the first antenna 116 and/or the electronics component 118 via one or more electrical connections extending through the lumen of the extension portion 106. For example, the proximal end portions of the electrical connections can be routed through the first connector 110 to the electronics component 118 on the electronics package 108. The electrical connections may comprise, for example, one or more wires, cables, traces, vias, and others extending through the extension portion 106 and lead body 104. The electrical connections can comprise a conductive material such as silver, copper, etc., and each electrical connection can be insulated along all or a portion of its length. In some embodiments, the device 100 includes a separate electrical connection for each conductive element 114. For example, in those embodiments in which the device 100 comprises eight conductive elements 114 (and other embodiments), the device 100 can comprise eight electrical connections, each extending through the lumen of the extension portion 106 from a proximal end at the electronics component 118 to a distal end at one of the conductive elements 114.

In some embodiments, the electronics component 118 comprise an application-specific integrated circuit (ASIC), a discrete electronic component, and/or an electrical connector. In these and other embodiments, the electronics component 118 can comprise, for example, processing and memory components (e.g., microcomputers, microprocessors, computers-on-a-chip, etc.), charge storage and/or delivery components (e.g., batteries, capacitors, electrical conductors) for receiving, accumulating, and/or delivering electrical energy, switching components (e.g., solid state, pulse-width modulation, etc.) for selection and/or control of the conductive elements 114. In some embodiments, the electronics component 118 comprise a data communications unit for communicating with an external device (such as external system 15) via a communication standard such as, but not limited to, near-field communication (NFC), infrared wireless, Bluetooth, ZigBee, Wi-Fi, inductive coupling, capacitive coupling, or any other suitable wireless communication standard. In some examples, the electronics component 118 include one or more processors having one or more computing components configured to control energy delivery via the conductive elements 114 and/or process energy and/or data received by the conductive elements 114 according to instructions stored in the memory. The memory may be a tangible, non-transitory computer-readable medium configured to store instructions executable by the one or more processors. For instance, the memory may be data storage that can be loaded with one or more of the software components executable by the one or more processors to achieve certain functions. In some examples, the functions may involve causing the conductive elements 114 to obtain data characterizing activity of a patient's muscles. In another example, the functions may involve processing data to determine one or more parameters of the data (e.g., a change in muscle activity, etc.). According to various embodiments, the electronics component 118 can comprise a wireless charging unit for providing power to other electronics component 118 of the device 100 and/or recharging a battery of the device 100 (if included).

The electronics package 108 can also be configured to wirelessly receive energy from a power source to power the treatment device 100. In some embodiments, the electronics package 108 comprises a first antenna 116 configured to wirelessly communicate with the external system 15. As shown in FIG. 2B, in some embodiments the electronics component 118 can be disposed in an opening at a central portion of the first antenna 116. In other embodiments, the electronics component 118 and antenna 116 may have other configurations and arrangements.

The second antenna 12 can be configured to emit an electromagnetic field to induce an electrical current in the first antenna 116, which can then be supplied to the electronics component 118 and/or conductive elements 114. In some embodiments, the first antenna 116 comprises a coil or multiple coils. For example, the first antenna 116 can comprise one or more coils disposed on a flexible substrate. The substrate can comprise a single substrate or multiple substrates secured to one another via adhesive materials. For instance, in some embodiments the substrate comprises multiple layers of a heat resistant polymer (such as polyimide) with adhesive material between adjacent layers. Whether comprising a single layer or multiple layers, the substrate can have one or more vias extending partially or completely through a thickness of the substrate, and one or more electrical connectors can extend through the vias to electrically couple certain electronic components of the electronics package 108, such as the first antenna 116 and/or the electronics component 118.

In some embodiments, the first antenna 116 comprises multiple coils. For example, the first antenna 116 can comprise a first coil at a first side of the substrate and a second coil at a second side of the substrate. This configuration can be susceptible to power losses due to substrate losses and parasitic capacitance between the multiple coils and between the individual coil turns. Substrate losses occur due to eddy currents in the substrate due to the non-zero resistance of the substrate material. Parasitic capacitance occurs when these adjacent components are at different voltages, creating an electric field that results in a stored charge. All circuit elements possess this internal capacitance, which can cause their behavior to depart from that of “ideal” circuit elements.

Advantageously, in some embodiments the first antenna 116 comprises a two-layer, pancake style coil configuration in which the top and bottom coils are configured in parallel. As a result, the coils can generate an equal or substantially equal induced voltage potential when subjected to an electromagnetic field. This can help to equalize the voltage of the coils during use, and has been shown to significantly reduce the parasitic capacitance of the first antenna 116. In this parallel coil configuration, the top and bottom coils are shorted together within each turn. This design has been found to retain the benefit of lower series resistance in a two-coil design while, at the same time, greatly reducing the parasitic capacitance and producing a high maximum power output. Additional details regarding the two-coil configuration can be found in U.S. application Ser. No. 16/866,523, filed May 4, 2020, which is incorporated by reference herein in its entirety. In some embodiments, the first antenna 116 comprises other coil arrangements, including a non-two layer design.

The first antenna 116 (or one or more portions thereof) can be flexible such that the first antenna 116 is able to conform at least partially to the patient's anatomy once implanted. In some embodiments, the first antenna 116 comprises an outer coating configured to encase and/or support the first antenna 116. The coating can comprise a biocompatible material such as, but not limited to, epoxy, urethane, silicone, or other biocompatible polymers. In some embodiments, the coating comprises multiple layers of distinct materials.

While being flexible, the lead 102 and/or one or more portions thereof (e.g., the elongate arms 104, the extension portion 106, etc.) can also be configured to maintain a desired shape. This feature can, for example, be facilitated by electrical conductors that electrically connect the conductive elements 114 carried by the lead body 104 to the electronics package 108, by an additional internal shape-maintaining (e.g., a metal, a shape memory alloy, etc.) support structure (not shown), by shape setting the substrate comprising the lead 102, etc. In any case, one or more portions of the lead 102 can have a physical property (e.g., ductility, elasticity, etc.) that enable the lead 102 to be manipulated into a desired shape or maintain a preset shape. Additionally or alternatively, the lead 102 and/or one or more portions thereof (e.g., the lead body 104, the extension portion 106, etc.) can be sufficiently flexible to at least partially conform to a patient's anatomy once implanted and/or to enhance patient comfort. For example, in some embodiments the extension portion 106 can be sufficiently flexible to bend back on itself such that at least the proximal portions of the arms 104 are positioned vertically above the electronics package 108 when the device 100 is implanted in a patient's under-chin region. (See, for example, FIG. 4B.) Additionally or alternatively, the elongate arms 104 can be sufficiently flexible to allow for bending between the first and second treatment zones 121, 123 such that the first treatment zone 121 is positioned in the under-chin region and the second treatment zone 123 is positioned along the patient's neck. In some embodiments, all or a portion of the extension portion 106 and/or all or a portion of the arms 104 can be pre-shaped so that, when implanted, the extension portion 106 and/or arms 104 are biased into assuming a geometry that facilitates positioning of the first and second treatment zones 121, 123 at desired treatment sites. Such bias(es) allows for better contact with the target tissue, more lead stability and hence better long-term performance of the lead.

The conductive elements 114 can be carried by the sidewall of the lead body 104. For example, the conductive elements 114 can be positioned on an outer surface of the sidewall and/or within a recessed portion of the sidewall. In some embodiments, one or more of the conductive elements 114 is positioned on an outer surface of the sidewall and extends at least partially around a circumference of the sidewall. The lumen of the lead body 104 can carry one or more electrical conductors that extend through the lumen of the lead body 104 and the lumen of the extension portion 106 from the conductive elements 114 to the electronics package 108. The sidewall can define one or more apertures through which an electrical connector can extend.

Each of the conductive elements 114 may comprise an electrode, an exposed portion of a conductive material, a printed conductive material, and other suitable forms. In some embodiments, one or more of the conductive elements 114 comprises a ring electrode. The conductive elements 114 can be crimped, welded, adhered to, or positioned over an outer surface and/or recessed portion of the lead body 104. Additionally or alternatively, each of the conductive elements 114 can be welded, soldered, crimped, or otherwise electrically coupled to a corresponding electrical connector. In some embodiments, one or more of the conductive elements 114 comprises a flexible conductive material disposed on the lead body 104 via printing, thin film deposition, or other suitable techniques. Each one of the conductive elements 114 can comprise any suitable conductive material including, but not limited to, platinum, iridium, silver, gold, nickel, titanium, copper, combinations thereof, and/or others. For example, one or more of the conductive elements 114 can be a ring electrode comprising a platinum iridium alloy. In some embodiments, one or more of the conductive elements 114 comprises a coating configured to improve biocompatibility, conductivity, corrosion resistance, surface roughness, durability, or other parameter(s) of the conductive element 114. As but one example, one or more of the conductive elements 114 can comprise a coating of titanium and nitride.

### III. Example Methods for Implanting the Treatment Devices of the Present Technology

In some instances, a surgical approach can be used to implant the treatment devices of the present technology. The surgical approach, for example, can access the patient's anatomy through a small incision on the underside of the patient's chin. Generally speaking, accessing the patient's anatomy from this location allows for other adjunctive procedures including, but not limited to, cervical liposuction (e.g., for effacement of platysmal banding), elevation of hyoid positioning, and mandibular distal bone advancement for aesthetic purposes and/or functionally repositioning the anterior lingual musculature.

An example placement of the treatment device 100 is shown in FIGS. 4A-4C. Even though these figures show the device 100 in its final, implanted location, FIGS. 4A-4C will be referenced in the following discussion of methods for implanting the device to help orient the reader with respect to the anatomy.

Implantation of the treatment device can begin by making a 1-2 cm transverse incision through the patient's skin and subcutaneous fat at or near the crease on the underside of the patient's chin. The physician then identifies and makes a 1-2 cm transverse dissection through the right and left platysma muscles and sub-platysmal fat. Next the physician identifies the anterior bellies of the right and left digastric muscles. Approaching from the medial ed ges, the physician inserts a finger or small blunt instrument into the fascial plane between the left and right anterior digastric bellies and the mylohyoid muscle and sweeps laterally (and potentially posteriorly and anteriorly) to make a small pocket that will eventually hold the electronics package 108 once implanted.

Next the physician may identify the midline raphe of the mylohyoid. In some cases it may be easier to identify the inferior portion of the raphe (i.e., the portion closer to the hyoid) first. The physician then dissects through the mylohyoid raphe, often times starting at the inferior portion and moving about 2.5-3 cm in the direction of the anterior mandible. Next, the physician dissects through the midline raphe of the geniohyoid to visualize the midline fat pad at the dorsal surface of the genioglossus (i.e., between a cranial surface of the geniohyoid and the dorsal surface of the genioglossus). For example, using a finger or soft blunt dissection tool, the physician may gently lift the cranial border of the geniohyoid in a direction away from the dorsal surface of the genioglossus to identify the lateral fat pad on the dorsal surface of the genioglossus as well as the distal arborization of the HGN.

At this stage, the physician may introduce a catheter (e.g., any tubular member, including a sheath, a needle (such as a Tuohy needle), a needle with an overlying sheath (including but not limited to a tear-away sheath)) containing one of the elongate arms 104 into the fascial plane between the geniohyoid and the genioglossus and advance the catheter posteriorly until nearing the hyoid, at which point the catheter can be routed inferiorly through the dissected raphes of the geniohyoid and mylohyoid, inferiorly past the hyoid (remaining anterior of the hyoid), and then laterally and inferiorly along a medial aspect of the patient's neck. As the catheter is advanced inferiorly beyond the hyoid, the catheter may be guided under (i.e., posterior to) the sternohyoid and advanced within a plane between the sternohyoid and sternothyroid. In some embodiments, the catheter and/or the elongate arms 104 can be pre-shaped in a certain geometry that biases the catheter and/or elongate arms 104 follow a desired path within the anatomy. For example, the catheter and/or elongate arms 104 can have one or more bends that cause the catheter and/or elongate arms 104 to curve laterally after extending through the raphe, then inferiorly again along a more lateral aspect of the neck. In some embodiments, the catheter is a steerable catheter.

In some embodiments, the catheter may include one or more stimulation electrodes along its outer surface that may be utilized prior to deployment of the treatment device 100 to locate an implantation position for the second treatment zone 123. Considering that the excitatory potential decreases inversely with the separation between the electrode and motor nerve, the electrode should be positioned at a point that is close to the region of the muscle where the major portion of the motor nerve fibers are located. This position or point is often referred to as the “motor point.” At the motor point, the stimulus amplitude required to fully activate the muscle is at its lowest value. The motor point can be identified by moving the catheter along the surface of the sternothyroid muscle and stimulating at various locations until finding the position that requires the least amplitude to fully activate the muscle. In some embodiments, a scope can be positioned in the airway to allow evaluation of the effects of stimulation from a more internal location. In those embodiments utilizing a needle, the tip of the needle can be used for stimulation. Once the motor point is located, the catheter can be withdrawn to allow the fixation member(s) at the second treatment zone 123 of the arm 104 expand and embed partially or completely within the sternothyroid muscle. Ultrasound, palpation, and/or other methods may be used during and/or after insertion to confirm location. The elongate arm 104 can be pulled proximally, just slightly, to further embed the fixation member(s). In some embodiments, the catheter comprises a peel-away catheter (i.e., the catheter sidewall is split along its longitudinal axis) to accommodate the bifurcation in the lead 102.

Next, the second treatment zone 123 on the other elongate arm 104 can be implanted on the sternothyroid other side of the neck following the same systems and methods described above. In some embodiments, only one of the elongate arms 104 includes a second treatment zone 123. In those cases, the treatment device 100 is configured to stimulate unilaterally (e.g., only the left or right sternothyroid).

It will be appreciated that the elongate arms 104 may follow a number of different paths through the anatomy between the first and second treatment zones 121, 123. As but one of several other examples, as shown in FIG. 4D, in some cases the elongate arms 104 may be routed laterally over the digastric muscles, then inferiorly along the sternothyroid.

With the second treatment zones 123 in position, the physician may test for functionality of the device 100 and proper localization of the conductive elements 114 relative to the sternothyroid and/or motor points by doing one or more test stimulations. During the test stimulations, the physician observes the sternothyroid and hyoid, specifically looking for airway stabilization (such as contraction of the sternothyroid and depression of the hyoid.

With the second treatment zones 123 implanted, the physician next implants the first treatment zones 121. The physician may grab one of the elongate arms 104 at or proximate to the first treatment zone 121 (e.g., via a forceps, pickup, or other instrument) and pushes the fixation element(s) at the first treatment zone 121 into the lateral fat pad at a position slightly more inferior than the last branch of the distal arborization of the corresponding right or left HGN. The physician then grabs the other of the elongate arms 104 at or proximate to the first treatment zone 121 (e.g., via a forceps, pickup, or other instrument) and pushes the fixation element(s) of that first treatment zone 121 into the lateral fat pad at a position slightly more inferior than the last branch of the distal arborization of the corresponding right or left HGN. Accordingly, the first treatment zone 121 of one of the elongate arms 104 is positioned at or proximate the right HGN and the first treatment zone 121 of the other elongate arm 104 is positioned at or proximate the left HGN. The first treatment zones 121 are configured to deliver energy to the HGNs to stimulate the genioglossus muscle.

It can be beneficial for the physician to confirm that the bifurcation joint is located substantially at the midline of the genioglossus. In some embodiments, the physician may secure the device 100 at one or more locations along the lead 102, including one or more locations along the extension portion 106 and/or one or more locations along the elongate arms 104. For example, in some cases the physician may use a tissue anchor (e.g., a surgical clip or other device) at the bifurcation between the left and right elongate arms 104. In some embodiments, the bifurcation joint includes a coupling portion that is configured to mate with the tissue anchor. The tissue anchor can beneficially secure the bifurcation joint in place and prevent movement superiorly or inferiorly.

With the elongate arms 104 in position, the physician may test for functionality of the device 100 and proper localization of the conductive elements 114 relative to the HGN by doing one or more test stimulations. During the test stimulations, the physician observes movement of the patient's tongue to assess whether the positions of the first treatment zones 121 need to be adjusted.

Once the position of the first treatment zones 121 is confirmed, the physician will close the mylohyoid and geniohyoid (e.g., using a suture) around the extension portion 106 of the device 100. In other words, the elongate arms 104 remain in the fascial plane between the genioglossus and the geniohyoid, but the extension portion 106 extends anteriorly and inferiorly away from the elongate arms 104, through the geniohyoid and mylohyoid. This prevents the lead 102 from moving laterally.

As best shown in FIG. 4A, the electronics package 108 is then positioned between the dorsal surface of the (now closed) mylohyoid and anterior bellies of the left and right digastric muscles. Next the physician closes the platysma, and finally stitches up the initial transverse incision.

### Conclusion

Although many of the embodiments are described above with respect to systems, devices, and methods for treating sleep apnea, the technology is applicable to other applications and/or other approaches. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-4D.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An implantable device for treating sleep apnea, comprising:
an electronics package; and
first and second elongate leads electrically coupled to the electronics package, wherein each of the first and second leads comprise:
a first conductive region configured to be implanted at a first treatment site at an under-chin region of the patient's head, wherein the first conductive region is configured to deliver a first electrical signal to the first treatment site to thereby activate the genioglossus muscle, and
a second conductive region configured to be implanted at a second treatment site at a neck region of the patient, wherein the second conductive region is configured to deliver a second electrical signal to the second treatment site to thereby activate an infrahyoid strap muscle.

2. The implantable device of claim 1, wherein the electronics package is configured to be implanted at an under-chin region of the patient's head.

3. The implantable device of claim 1, wherein the first electrical signal is configured to activate the genioglossus muscle via electrical stimulation of the hypoglossal nerve.

4. The implantable device of claim 1, wherein the second electrical signal is configured to directly stimulate the infrahyoid strap muscle.

5. The implantable device of claim 1, wherein the second electrical signal is configured to stimulate the infrahyoid strap muscle via stimulation of the ansa cervicalis.

6. The implantable device of claim 1, wherein the second conductive region is distal of the first conductive region along a longitudinal axis of the respective first and second elongate leads.

7. The implantable device of claim 1, wherein the first and second elongate leads are configured to be implanted such that the first conductive regions are aligned with and extend along a left hypoglossal nerve and a right hypoglossal nerve, respectively.

8. The implantable device of claim 1, wherein the first and second elongate leads are configured to be implanted such that the second conductive regions are at or within a left sternothyroid muscle and a right sternothyroid muscle, respectively.

9. The implantable device of claim 1, wherein the second treatment site is on or within the sternothyroid muscle.

10. The implantable device of claim 1, further comprising an extension having a proximal end portion configured to be coupled to the electronics package and a distal end portion, and wherein proximal end portions of each of the first and second elongate leads are coupled to the distal end portion of the extension.

11. The implantable device of claim 1, wherein each of the first and second elongate leads extends between a proximal end portion at the electronics package and a distal end portion.

12. The implantable device of claim 1, further comprising a first fixation element disposed at or near the first conductive region of the first elongate lead and a second fixation element disposed at or near the first conductive region of the second conductive lead, wherein each of the first and second fixation elements is configured to anchor the corresponding first or second elongate lead to tissue at the first treatment sites.

13. The implantable device of claim 1, further comprising a first fixation element disposed at or near the second conductive region of the first elongate lead and a second fixation element disposed at or near the second conductive region of the second conductive lead, wherein each of the first and second fixation elements is configured to anchor the corresponding first or second elongate lead to tissue at the second treatment sites.

14. An implantable device for treating sleep apnea, comprising:
an electronics package;
a first conductive region electrically coupled to the electronics package and configured to be implanted at a first treatment site at an under-chin region of the patient's head, wherein the first conductive region is configured to deliver a first electrical signal to the first treatment site to thereby activate the genioglossus muscle; and
a second conductive region electrically coupled to the electronics package and configured to be implanted at a second treatment site at a neck region of the patient, wherein the second conductive region is configured to deliver a second electrical signal to the second treatment site to thereby activate an infrahyoid strap muscle.
